(19) United States
(12) Patent Application Publication
(10) Pub. No.: US 2006/0069362 A1
(43) Pub. Date: Mar. 30, 2006

(54) Absorbent article with indicator

(75) Inventors: Thomas Walter Odorzynski, Green Bay, WI (US); Brenda Marie Nelson, Appleton, WI (US); Kellie Marie Goodrich, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/953,993

(22) Filed: Sep. 28, 2004

(51) Int. Cl. A61F 13/15

(52) U.S. Cl. 604/361

(57) ABSTRACT

An absorbent article includes a liquid permeable bodyside liner, an outer cover, and an absorbent body located between the bodyside liner and the outer cover. The absorbent body comprises at least one colorant. The colorant, when wetted with a liquid, becomes visible so as to identify the presence of the liquid. The colorant may also detect substances in the liquid. The colorant can detect small volumes of liquid and volumes of liquids deposited outside the target area.

### BACKGROUND

Various absorbent articles, such as disposable diapers, are commercially available with wetness indicators. The indicators identify when a diaper has received a substantial quantity of liquid and should be changed. For example, these indicators can include the appearance or disappearance of a design on the outer surface of the diaper that is visually identifiable at a distance. Other absorbent articles include indicators that identify the presence of various substances in the urine, such as protein.

However, newborn babies generally have very small urine discharges of about 10 to about 15 cubic centimeters (cc). Due to the advanced technology of disposable diapers and their ability to keep the baby dry and clean, it can be difficult to detect whether any insult has occurred. As a result, it can be difficult for parents to monitor the child's quantity and frequency of urination. This information may be helpful in diagnosing whether an infant/child is dehydrated (a common cause of newborn babies being readmitted to the hospital after birth). Additionally, it can be difficult to collect a urine sample from a small baby to determine whether various substances exist in the urine. Hence, there is a need to be able to identify whether or not small insult(s) have occurred and to determine whether various substances exist in urine.

### BRIEF SUMMARY

Disclosed herein are absorbent articles and absorbent article systems. In one embodiment, an absorbent article can comprise a liquid permeable bodyside liner, an outer cover, and an absorbent body located between the bodyside liner and the outer cover. The absorbent body can comprise a first colorant that becomes visible when wetted with urine so as to identify the presence of the urine. In various embodiments, the colorant can become visible in the presence of about 1 to 10 cc of urine. In other embodiments, the colorant can become visible in the presence of about 1 to 5 cc of urine. In various embodiments, the colorant may be disposed over greater than or equal to 50% of the absorbent body area. In yet other embodiments, the absorbent body may further include superabsorbent particles and absorbent fibers intermixed with the colorant.

In another embodiment, an absorbent article can comprise a liquid permeable bodyside liner, an outer cover, an absorbent body located between the bodyside liner and the outer cover, and a first colorant. The first colorant becomes visible in the presence of a substance in urine. The substance can be selected from the group consisting of blood, protein, nitrate, ketones, glucose, leukocytes, urobilinogen, bilirubin and combinations comprising at least one of the foregoing substances. In other embodiments, the absorbent body may comprise one or more second colorants. The one or more second colorants may become visible in the presence of a substance in the urine different than the substance in the urine that makes the first colorant visible.

In various embodiments, the colorant may be encapsulated in an encapsulant that is soluble in urine.

In yet another embodiment, an absorbent article can comprise a colorant having a first state when the absorbent article is dry and a second state when the absorbent article is wetted with a volume of urine. The second state can be visible and can be indicative of the volume of the urine. The absorbent article can include a liquid permeable bodyside liner, an outer cover, and an absorbent body. The absorbent body may comprise the colorant. The absorbent body may also include cellulose fibers and superabsorbent particles intermixed with the colorant.

In one embodiment, an absorbent article system can comprise a liquid permeable bodyside liner, an outer cover, an absorbent body located between the bodyside liner and the outer cover, and a correlater. The absorbent body comprises a colorant that, when wetted, becomes visible and defines a visible area. The correlater can be useful in estimating a volume of liquid received by the absorbent body by comparing the visible area to the correlater. The correlater may be selected from the group consisting of a graph, a chart, a diagram, a table, a design, and combinations comprising at least one of the foregoing.

In various embodiments, the absorbent article may include a surge material.

The above described and other features are exemplified by the following figures and detailed description.

### BRIEF DESCRIPTION OF DRAWINGS

Refer now to the figures, which are exemplary, not limiting, and wherein like elements are numbered alike.

FIG. 1 representatively shows a partially cut away, top plan view of an absorbent article in a stretched and laid flat condition with the surface of the article that contacts the skin of the wearer facing the viewer.

FIG. 2 representatively shows a sectional view of the absorbent article of FIG. 1 taken along line 2-2.

FIG. 3 representatively illustrates points A and B referenced in the example.

### DETAILED DESCRIPTION

It is noted that all ranges disclosed herein are inclusive and combinable (e.g., ranges of “up to about 25 wt %, or, more specifically about 5 wt % to about 20 wt %” is inclusive of the endpoints and all intermediate values of the ranges of “about 5 wt % to about 25 wt %,” etc.). The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced items. Unless defined otherwise, technical and scientific terms used herein have the same meaning as is commonly understood by one of skill in the art to which this invention belongs. The modifier “about” used in connection with a quantity is inclusive of the stated value and has the meaning dictated by the context, (e.g., includes the degree of error associated with measurement of the particular quantity).

Disclosed herein are absorbent articles and systems for determining the existence of an insult, for estimating a volume of a liquid in absorbent articles, and for identifying one or more substances in urine. The absorbent articles can comprise a colorant having a first state when the absorbent article is dry and a second state when the absorbent article is wetted with a volume of a liquid. A difference between the first state and the second state can indicate the existence of an insult, the volume of the liquid, and/or the existence of substance in the urine (e.g., blood, protein, etc.). Therefore, the absorbent article can comprise one or more colors, desirably encapsulated colorants. If an encapsulant is employed, it would dissolve upon contact with a particular liquid (e.g., urine, water, a substance in the liquid, and/or the like), thereby releasing the colorant, and changing the color to illustrate a difference from the first state to the second state. In general, the greater the volume of liquid present, the greater the release of colorant, thus providing a relative visual indication of the volume of the liquid. In addition, or alternatively, the colorant can have a reaction with the insult or a component thereof, such that a color change occurs. The color change could start with a color (blue, green, yellow, etc.) and become colorless such that only the color of the diaper is seen. Alternatively, the color change could start with one color and change to another (e.g., from blue to green). In yet another alternative, the colorant could be white or essentially colorless and change to one or more colors upon contact with a particular liquid, such as urine. Possible substances in urine that may be detected include blood, protein, nitrates, ketones, glucose, leukocytes, urobilinogen, bilirubin and combinations comprising at least one of the foregoing substances.

The colorant is desirably a color visible to the human eye, thereby enabling identification of an insult without additional equipment (e.g., ultraviolet light, or the like). Also, due to the use of colorants in an absorbent article, it is desirable that the color be a material approved by the U.S. Food and Drug Administration (FDA) for use in food. Optionally, the colors may be various colors corresponding to different color hues of the same base color. For example, the colors could be a progression from light blue to dark blue wherein the hue of the color indicates the volume of the insult, for example.

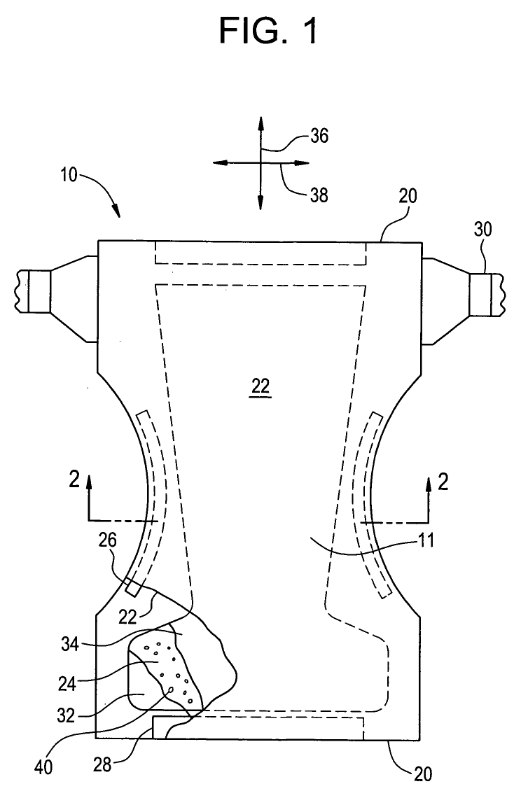

Referring now to FIG. 1, in one embodiment, the absorbent article is a disposable diaper 10 comprising a backsheet or outer cover 20, a liquid permeable topsheet or bodyside liner 22 positioned in facing relation with the outer cover 20, and an absorbent body 24, such as an absorbent pad, which is located between the bodyside liner 22 and the outer cover 20. The outer cover 20 defines a length and a width that, in the illustrated aspect, coincide with the length and width of the diaper 10. The absorbent body 24 generally defines a length and width that are less than the length and width of the outer cover 20, respectively. Thus, marginal portions of the diaper 10, such as marginal sections of the outer cover 20, may extend past the terminal edges of the absorbent body 24. In the illustrated aspects, for example, the outer cover 20 extends outwardly beyond the terminal marginal edges of the absorbent body 24 to form side margins and end margins of the diaper 10. The bodyside liner 22 is generally coextensive with the outer cover 20 but may optionally cover an area that is larger or smaller than the area of the outer cover 20, as desired. In other words, the bodyside liner 22 is connected in superposed relation to the outer cover 20. The outer cover 20 and bodyside liner 22 are intended to face the garment and body of the wearer, respectively, while in use.

To provide improved fit and to help reduce leakage of body exudates from the diaper 10, the diaper side margins and end margins can be elasticized with suitable elastic members, such as single or multiple strands of elastic. The elastic strands may be composed of natural or synthetic rubber and may optionally be heat shrinkable or heat elasticizable. For example, as representatively illustrated in FIG. 1, the diaper 10 may include leg elastics 26 that are constructed to operably gather and shirr the side margins of the diaper 10 to provide elasticized leg bands which can closely fit around the legs of the wearer to reduce leakage and provide improved comfort and appearance. Similarly, waist elastics 28 can be employed to elasticize the end margins of the diaper 10 to provide elasticized waists. The waist elastics 28 are configured to operably gather and shirr the waist sections to provide a resilient comfortably close fit around the waist of the wearer. In the illustrated aspects, the elastic members are illustrated in their uncontracted, stretched condition for the purpose of clarity.

Fastening means, such as hook and loop fasteners 30, can be employed to secure the diaper 10 on a wearer. Alternatively, other fastening means, such as buttons, pins, snaps, adhesive tape fasteners, cohesives, mushroom-and-loop fasteners, a belt, and so forth, as well as combinations comprising at least one of the foregoing fasteners may be employed. Additionally, more than two fasteners can be provided, particularly if the diaper 10 is to be provided in a prefastened configuration.

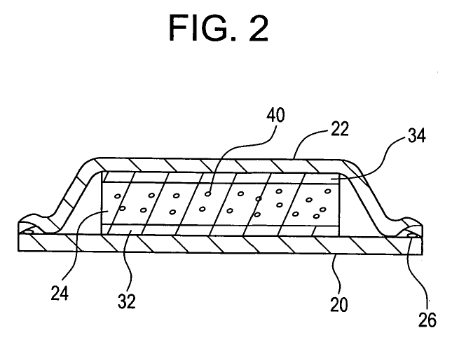

The diaper 10 may further include other layers between the absorbent body 24 and the bodyside liner 22 or outer cover 20. For example, as representatively illustrated in FIGS. 1 and 2, the diaper 10 may include a ventilation layer 32 located between the absorbent body 24 and the outer cover 20 to insulate the outer cover 20 from the absorbent body 24, to improve air circulation and to effectively reduce the dampness of the garment facing surface of the outer cover 20. The ventilation layer 32 may also assist in distributing fluid exudates to portions of the absorbent body 24 that do not directly receive the insult. The diaper 10 may also include a surge management layer 34 located between the bodyside liner 22 and the absorbent body 24 to prevent pooling of the fluid exudates and further improve air exchange and distribution of the fluid exudates within the diaper 10.

The diaper 10 may be of various suitable shapes. For example, the diaper may have an overall rectangular shape, T-shape or an approximately hourglass shape. In the shown aspect, the diaper 10 has a generally I-shape. The diaper 10 further defines a longitudinal direction 36 and a lateral direction 38. Other suitable diaper components that may be incorporated on absorbent articles include containment flaps, waist flaps, elastomeric side panels, and the like. Examples of possible diaper configurations are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996 to Hanson et al.

The various components of the diaper 10 are integrally assembled together employing various types of attachment mechanisms that do not adversely effect the colorant (and encapsulation, if present), such as adhesive, sonic bonds, thermal bonds, and so forth, as well as combinations comprising at least one of foregoing mechanisms. In the shown aspect, for example, the bodyside liner 22 and outer cover 20 are assembled together and to the absorbent body 24 with lines of adhesive, such as a hot melt, pressure-sensitive adhesive. Similarly, other diaper components, such as the elastic members 26 and 28, fastening members 30, and ventilation and surge layers 32 and 34 may be assembled into the diaper 10 by employing the above-identified attachment mechanisms.

The outer cover 20 of the diaper 10 can comprise any material used for such applications, such as a substantially vapor permeable material. The permeability of the outer cover 20 can be configured to enhance the breathability of the diaper 10 and to reduce the hydration of the wearer's skin during use without allowing excessive condensation of vapor, such as urine, on the garment facing surface of the outer cover 20 that can undesirably dampen the wearer's clothes. The outer cover 20 can be constructed to be permeable to at least water vapor and can have a water vapor transmission rate of greater than or equal to about 1,000 grams per square meter per 24 hours (g/m2/24 hr). For example, the outer cover 20 may define a water vapor transmission rate of about 1,000 to about 6,000 g/m2/24 hr.

The outer cover 20 is also desirably substantially liquid impermeable. For example, the outer cover 20 may be constructed to provide a hydrohead value of greater than or equal to about 60 centimeters (cm), or, more specifically, greater than or equal to about 80 cm, and even more specifically, greater than or equal to about 100 cm. A suitable technique for determining the resistance of a material to liquid penetration is Federal Test Method Standard (FTMS) 191 Method 5514, dated Dec. 31, 1968.

As stated above, the outer cover 20 can comprise any material used for such applications, and desirably comprises materials that either directly provide the above desired levels of liquid impermeability and air permeability and/or materials that can be modified or treated in some manner to provide such levels. The outer cover 20 can be a nonwoven fibrous web constructed to provide the required level of liquid impermeability. For example, a nonwoven web comprising spunbond and/or meltblown polymer fibers may be selectively treated with a water repellent coating and/or laminated with a liquid impermeable, vapor permeable polymer film to provide the outer cover 20. In another embodiment, the outer cover 20 can include a nonwoven web comprising a plurality of randomly deposited hydrophobic thermoplastic meltblown fibers that are sufficiently bonded or otherwise connected to one another to provide a substantially vapor permeable and substantially liquid impermeable web. The outer cover 20 may also include a vapor permeable nonwoven layer that has been partially coated or otherwise configured to provide liquid impermeability in selected areas. In yet another example, the outer cover 20 is provided by an extensible material. Further, the outer cover 20 material can have stretch in the longitudinal 36 and/or lateral 38 directions. When the outer cover 20 is made from extensible or stretchable materials, the diaper 10 provides additional benefits to the wearer including improved fit.

The bodyside liner 22, employed to help isolate the wearer's skin from liquids held in the absorbent body 24, can define a compliant, soft feeling, nonirritating to the wearer's skin bodyfacing surface. Further, the bodyside liner 22 can be less hydrophilic than the absorbent body 24, to present a relatively dry surface to the wearer, and can be sufficiently porous to be liquid permeable, permitting liquid to readily penetrate through its thickness. A suitable bodyside liner 22 can be manufactured from a wide selection of web materials, such as porous foams, reticulated foams, apertured plastic films, natural fibers (for example, wood or cotton fibers), synthetic fibers (for example, polyester or polypropylene fibers), and the like, as well as a combination of materials comprising at least one of the foregoing materials.

Various woven and nonwoven fabrics can be used for the bodyside liner 22. For example, the bodyside liner 22 can comprise a meltblown or spunbond web (e.g., of polyolefin fibers), a bonded-carded web (e.g., of natural and/or synthetic fibers), a substantially hydrophobic material (e.g., treated with a surfactant or otherwise processed to impart a desired level of wetability and hydrophilicity), and the like, as well as combinations comprising at least one of the foregoing. For example, the bodyside liner 22 can comprise a nonwoven, spunbond, polypropylene fabric, optionally comprising about 2.8 to about 3.2 denier fibers formed into a web having a basis weight of about 22 grams per square meter (g/m²) and a density of about 0.06 gram per cubic centimeter (g/cc).

The absorbent body 24 of the diaper 10 can comprise a matrix of hydrophilic fibers, such as a fibrous web of cellulosic fibers, mixed with particles of a high-absorbency material (such as the material commonly known as superabsorbent material). The wood pulp fluff may be exchanged with synthetic, polymeric, meltblown fibers, and the like, as well as a combination comprising at least one of the foregoing. The superabsorbent particles can be substantially homogeneously mixed with the hydrophilic fibers or may be nonuniformly mixed. Alternatively, the absorbent body 24 can include a laminate of fibrous webs and superabsorbent material and/or a suitable matrix for maintaining the superabsorbent material in a localized area.

A colorant 40 can be disposed in the absorbent body 24, e.g., as shown in FIGS. 1 and 2. The colorant 40 can be disposed in the absorbent body 24 in such a way that areas of the absorbent article that may receive an insult are adjacent to the colorant such that the colorant can indicate the presence and/or propert(ies) of the insult. For example, greater than or equal to about 90 percent of an inner surface of the bodyside liner (i.e., the surface of the bodyside liner facing the outer cover 20) can be disposed adjacent to the colorant. The colorant 40 can be mixed, adsorbed, absorbed, blended, and/or otherwise disposed within and/or coated on the absorbent body 24. As such, the colorant 40 may be spread across substantially all of the absorbent body 24 area (the absorbent body area defined by the plain created by longitudinal direction 36 and lateral direction 38).

The colorant 40 can comprise any material that can indicate the desired property when an insult having a volume as small as about 1 cc is received. The colorant can comprise a dye, such as bromocresol green; m-cresol purple; cresol red; chlorophenol red; bromothymol blue; bromopyrogallol red; bromoxylenol blue; methylene blue; bromophenol blue; monoazo dyes (such as acid alizarin violet N); monoazo pyrazoline dyes (such as acid yellow 34); diazo dyes (such as acid black 24); anthraquinone dyes (such as acid black 48); amphoteric anthraquinone dyes (such as acid blue 45); triphenylmethane dyes (such as acid fuchsin); phthalein type dyes (such as o-cresolphthalein); xanthene dyes (such as 2′,7′-dichlorofluorescein eosin B); heterocyclic acridine aromatics (such as acridine orange); diphenylmethane dyes (such as auramine O); triphenylmethane dyes (such as basic fuchsin); cationic thiazine dyes (such as azure C); cationic anthraquinone dyes (such as basic blue 47); phthalocyanine type dyes (such as copper phthalocyanine); quaternized phthalocyanine type dyes (such as alcec blue); cationic polymethine dyes (such as astrazon orange G); anthraquinone type (such as alizarin); the neutral complex dyes (such as azure A eosinate); the terpene type dyes (such as trans-beta-carotene); and so forth, as well as combinations comprising at least one of the foregoing dyes. Desirably, the colorant(s) are U.S. FDA approved.

The colorant 40 can be encapsulated in a material that is soluble in urine. Suitable urine or water soluble materials include, but are not limited to, cellulose-based polymeric materials (such as ethyl cellulose); carbohydrate-based materials (such as starches, sugars, and materials derived therefrom, e.g., dextrins and cyclodextrins); and so forth, as well as combinations comprising at least one of the foregoing materials. When the colorant 40 is contacted by urine, the encapsulating material dissolves releasing the dye contained therein. The visual presence of the dye indicates that urine is present. The amount of urine necessary to create a visual indication is at least about 1 to 15 cc, 1 to 10 cc, 1 to 5 cc, or at least 1 cc.

The colorant 40 can alternatively or additionally comprise one or more materials that can indicate the presence of a substance in urine. This technology is currently available in urine test strips commonly used in the medical profession. Exemplary materials are set forth in the Compendium Visual Urinalysis with Test Strips, by Dr. Ruth Draisbach et al., Roche Diagnostics GmbH. For example, pH levels in the urine may be indicated by using methyl red, bromthymol blue, and phenolphthalein. The pH range 5 to 9 yields a color gradation from orange to yellow to green to blue. Leukocytes may be indicated with indoxyl ester and a diazonium salt which yields a violet color. Nitrates in the urine may be indicated with aromatic amine sulfanilamide and 3-hydrozyl-1,2,3,4-tretrahydrobenzo-(h)-quinoline which yields a red color. Protein in the urine may be indicated by 3′,3″,5′,5″-tetrachlorophenol-3,4,5,6-tetrabromosulfonphthalien yielding a color change from yellow to green. Glucose may be indicated with 3,3′,5,5′-tetramethylbenzidine yielding a blue-green dye. Ketones may be indicated with sodium nitroprusside to give a violet color. Urobilinogen may be indicated with p-methoxybenzenediazonium fluoroborate (diazonium salt) to yield a red color. Bilirubin may be detected using 2,6-dichlorobenzenediazonium fluoroborate (diazonium salt) to form a red-violet color. Blood may be indicated with 3,3′,5,5′-tetramethylbenzidine and 2,5-dimethylhexane-2,5-dihydroperoxide to yield a blue-green color. Any combination of these materials may be encapsulated in a water or urine soluble encapsulant. The encapsulated materials may be dispersed throughout the absorbent structure or may be concentrated in the target zone. More than one colorant may be included in any single absorbent article. In various embodiments, one or more colorants indicating one or more substances in the urine may be intermixed in the absorbent body. For example, a first colorant may indicate the presence of glucose, a second colorant may indicate the presence of ketones, and a third colorant may indicate the presence of urine. All three colorants may be intermixed with cellulose and superabsorbent in the absorbent body of an absorbent article.

In order to ensure the identification of all insults, including small and “off target” insults, it is desirable to dispose the colorant 40 in the absorbent body 24. Desirably, sufficient colorant 40 is disposed on a surface of the absorbent body 24 (e.g., the surface adjacent the bodyside liner 22 and opposite the outer cover 20) and/or throughout the absorbent body 24 to ensure identification of any insults by the wearer. The absorbent body 24 has an area in the plane defined by the longitudinal direction 36 and the lateral direction 38, and the colorant can be disposed in greater than or equal to about 50% of the area. Alternatively, the colorant 40 may be disposed over greater than about 60%, greater than about 70%, greater than about 80%, greater than about 90% percent, or greater than about 95% percent of the area of the absorbent body 24. In some embodiments, the colorant 40 may be disposed over substantially 100% of the area of the absorbent body 24. When the colorant 40 is widely dispersed, the urine insult is unlikely to “miss” the colorant. Also, the wide dispersement of colorant 40 may indicate multiple insults at different locations. This information may be useful in determining the frequency of insults. The colorant 40 can be present in an amount of less than or equal to about 0.5 weight percent (wt %), or, more specifically, about 0.05 w % to about 0.25 wt %, based upon the total weight of the absorbent body 24.

In addition to the colorant 40, the absorbent body 24 can comprise a fibrous web (e.g., comprising cellulosic fibers) and a superabsorbent material. When the absorbent body 24 comprises a combination of hydrophilic fibers and high-absorbency particles, the hydrophilic fibers and high-absorbency particles can form an average basis weight for the absorbent body 24 that can be about 400 grams per square meter (g/m²) to about 900 g/m², or, more specifically, about 500 g/m² to about 800 g/m², and even more specifically, about 550 g/m² to about 750 g/m².

The high-absorbency material (e.g., superabsorbent) can be natural, synthetic, and modified natural polymers and materials; inorganic materials (such as silica gels); organic compounds (such as crosslinked polymers); and the like, as well as combinations comprising at least one of the foregoing. The term “crosslinked” refers to methods for effectively rendering normally water-soluble materials substantially water insoluble but swellable. Such methods include, but are not limited to, physical entanglement, crystalline domains, covalent bonds, ionic complexes and associations, hydrophilic associations such as hydrogen bonding, and/or hydrophobic associations or Van der Waals forces. Examples of high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and the like, as well as copolymers and combinations comprising at least one of the foregoing. Further polymers suitable for use in the absorbent body 24 include, but are not limited to, polymers (natural and modified natural), such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and so forth. Mixtures of natural and wholly or partially synthetic absorbent polymers can also be useful. Similarly useful are various copolymers and, combinations comprising at least one of any of the above high-absorbency materials. An example of high-absorbency material is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

The high absorbency material may be in any of a wide variety of geometric forms. Generally, it is preferred that the high absorbency material be in the form of discrete particles. However, the high absorbency material may also be in the form of fibers, flakes, rods, spheres, needles, particles, or the like, as well as combinations comprising at least one of the foregoing. In general, the high absorbency material is present in the absorbent body 24 in an amount of greater than or equal to about 5 weight percent (wt %), or, more specifically greater than or equal to about 30 wt %, and even more specifically, greater than or equal to about 50 wt % based on a total weight of the absorbent body 24. For example, in a particular aspect, the absorbent body 24 may include a laminate which includes greater than or equal to about 50 wt % , or, more specifically, greater than or equal to about 70 wt % of high-absorbency material overwrapped by a fibrous web or other suitable material for maintaining the high-absorbency material in a localized area.

Optionally, the absorbent body 24 may further comprise a support (e.g., a substantially hydrophilic tissue or nonwoven wrapsheet (not illustrated)) to help maintain the integrity of the structure of the absorbent body 24. The tissue wrapsheet can be placed about the web/sheet of high-absorbency material and/or fibers, optionally over at least one or both major facing surfaces thereof. The tissue wrapsheet can comprise an absorbent cellulosic material, such as creped wadding or a high wet-strength tissue. The tissue wrapsheet can optionally be configured to provide a wicking layer that helps to rapidly distribute liquid over the mass of absorbent fibers constituting the absorbent body 24. If this support is employed, the colorant 40 may optionally be disposed in the support, on the side of the absorbent body 24 opposite the outer cover 20.

Due to the thinness of absorbent body 24 and the high absorbency material within the absorbent body 24, the liquid uptake rates of the absorbent body 24, by itself, may be too low, or may not be adequately sustained over multiple insults of liquid into the absorbent body 24. To improve the overall liquid uptake and air exchange, the diaper 10 may further include a porous, liquid-permeable layer of surge management material 34, as representatively illustrated in FIG. 1. The surge management layer 34 is typically less hydrophilic than the absorbent body 24, and can have an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the diaper 10 positioned against the wearer's skin, thereby reducing the feeling of wetness by the wearer. The structure of the surge management layer 34 can also enhances the air exchange within the diaper 10.

Various woven and nonwoven fabrics can be used to construct the surge management layer 34. For example, the surge management layer 34 may be a layer comprising a meltblown or spunbond web of synthetic fibers (such as polyolefin fibers); a bonded-carded-web or an airlaid web comprising, for example, natural and/or synthetic fibers; hydrophobic material that is optionally treated with a surfactant or otherwise processed to impart a desired level of wetability and hydrophilicity; and the like, as well as combinations comprising at least one of the foregoing. The bonded-carded-web can, for example, be a thermally bonded web that is bonded using low melt binder fibers, powder, and/or adhesive. The layer can optionally include a mixture of different fibers. For example, the surge management layer 34 can comprise a hydrophobic, nonwoven material having a basis weight of about 30 to about 120 g/m².

As representatively illustrated in FIG. 1, the diaper 10 can optionally also include a ventilation layer 32 located between the outer cover 20 and the absorbent body 24. The ventilation layer 32 can serve to facilitate the movement of air within and through the diaper 10 and to prevent the outer cover 20 from being in surface to surface contact with at least a portion of the absorbent body 24. The ventilation layer 32 can serve as a conduit through which air and water vapor can move from the absorbent body 24 through the vapor permeable outer cover 20.

The ventilation layer 32 may be formed from materials described above as being suitable for the surge management layer 34 such as nonwoven, (e.g., spunbond, meltblown, carded, and the like), woven, knitted fibrous webs, and the like, comprising natural fibers, synthetic polymeric fibers, and/or the like. Suitable fibers include, for example, acrylic fibers, polyolefin fibers, polyester fibers, and the like, as well as blends comprising at least one of the foregoing fibers. The ventilation layer 32 may also be formed from a porous foam material such as an open-celled polyolefin foam, a reticulated polyurethane foam, and the like. The ventilation layer 32 may include a single layer of material or a composite of two or more layers of material. For example, the ventilation layer 32 can include a hydrophobic, nonwoven material having a thickness of greater than or equal to about 0.10 centimeters (cm) determined under a restraining pressure of 0.34 kiloPascals (kPa) and a basis weight of about 20 g/m² to about 120 g/m². For example, the ventilation layer 32 may comprise a bonded-carded-web, nonwoven fabric with bicomponent fibers, and defining an overall basis weight of about 83 g/m². The ventilation layer 32 in such a configuration can be a homogeneous blend comprising about 60 wt % polyethylene/polyester (PE/PET), sheath-core bicomponent fibers that have a fiber denier (d) of about 3 d, and about 40 wt % single component polyester fibers that have a fiber denier of about 6 d and fiber lengths of about 3.8 cm to about 5.08 cm.

The ventilation layer 32 can be any desired shape. Suitable shapes include for example, circular, rectangular, triangular, trapezoidal, oblong, dog-boned, hourglass-shaped, oval, and the like. The ventilation layer 32 may extend beyond, completely over, or partially over a side of the absorbent body 24 disposed opposite the bodyside liner 22. For example, the ventilation layer 32 may suitably be located over the intermediate section 16 of the diaper 10 and be substantially centered side-to-side with respect to the longitudinal centerline 36 of the diaper 10. It is generally desired that the entire absorbent body 24 be overlaid with the ventilation layer 32 to prevent substantially all surface to surface contact between the outer cover 20 and the absorbent body 24. In the illustrated figures, the ventilation layer 32 is coextensive with the absorbent body 24. This allows for a high degree of air exchange with minimal dampness on the garment facing surface of the outer cover 20.

In the illustrated figures, the ventilation layer 32 is arranged in a direct, contacting liquid communication with the absorbent body 24. The ventilation layer 32 may be operably connected to the outer cover 20 with a pattern of adhesive, such as a swirl adhesive pattern. In addition, the ventilation layer 32 may be operably connected to the absorbent body 24 with a pattern of adhesive. The amount of adhesive add-on should be sufficient to provide the desired levels of bonding, but should be low enough to avoid excessively restricting the movement of air and vapor from the absorbent body 24 and through the outer cover 20.

In various embodiments, the colorant may be employed to estimate a quantity of an insult. If the article comprises a surge management material that is designed to spread the insult laterally and longitudinally, the depth of the insult becomes somewhat uniform and the volume can be estimated by using the area of the insult in conjunction with a size correlater. The size correlator can be a table, chart, graph, design, diagram, or the like, as well as combinations comprising at least one of the foregoing. The correlater can be marked on the article itself, on the article packaging, on an insert provided with the article, and/or otherwise be made available (e.g., via the internet). For example, when the article receives an insult, the insult releases the colorant and causes a color change in the article. The volume of urine can be estimated by measuring the area of the stain and comparing it to the correlater. The color of the insult can also be associated with a volume because the greater the volume, the greater the number of encapsulated colorants rupturing and becoming visible. It is noted that removal of color from an area can also be used to determine the presence of a subsequent insult.

It is further noted that the frequency of insults, which can be useful information, can be determined, e.g., by the presence of multiple color (or colorless, accordingly) areas; e.g., “stains”. For example, if multiple insults contact the absorbent article at different locations, each will produce a color change that can be identified. Depending upon the amount of time the absorbent article was worn and the number of insults identified, the frequency of insults can be determined.

The disclosure is further illustrated by the following non-limiting example.

### EXAMPLE

A colorant comprising 20 wt % of methylene blue and 80 wt % of water soluble starch was mixed into Stockhausen Favor 9394 superabsorbent material in a ratio of 5 grams colorant to 95 grams superabsorbent. This mixture was combined with CR 1654 pulp at a ratio of 50 wt % colorized superabsorbent mixture to 50 wt % pulp to produce an absorbent body. The final composition of the absorbent body was 50 wt % of pulp, 47.5 wt % of superabsorbent material and 2.5 wt % of the colorant, based upon the total weight of the absorbent body. This absorbent body was white in the dry state before receiving an insult.

The absorbent body was insulted with 10 cubic centimeters (cc) saline solution (0.9 gram per liter). The insult resulted in a blue area becoming visible on the absorbent body. A second experiment was carried out wherein an absorbent body, as described above, was insulted with 1 cc of saline solution (0.9 gram per liter). A blue area again appeared on the absorbent body demonstrating that even with insults as small as 1 cc, the presence of the insult can be identified.

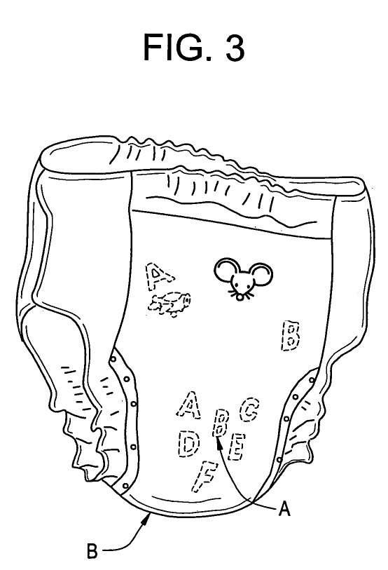

Three commercially available absorbent products with backsheet indicators were also observed (PAMPERS EASY UPS size 3T made by Procter & Gamble, MOONY OSHIRI PURE size A-newborn made by Uni-Charm, and MERRIES size small made by Kao). The PAMPERS indicator included graphics in the front crotch region of the product that faded when wet. The MOONY products had a separate strip printed with graphics located between the absorbent core and the backsheet that ran the entire length of the product. The strip graphics became purple/blue and thus more visible when wet. The MERRIES products had three yellow stripes that ran down the center of the product and extended the entire length of the product. The stripes were located between the absorbent core and the backsheet. These stripes turned blue when wet. The first observation involved adding an insult of 15 cc of saline solution (0.9 gram per liter) to the liner side of each absorbent product directly over the backsheet graphics. The point of insult was opposite location A as illustrated in FIG. 3. The observation was repeated three times for each commercial brand using a new article each time. The observations were made 1 minute after the product was insulted. In each case, there was visual indication that an insult had occurred. The visual indication was either fading graphics or appearing color depending on the product.

A second observation involved adding a 5 cc saline solution (0.9 gram per liter) insult to new samples in the same location as before. This was repeated three times per brand using a new product each time. No visual change in the graphics was observed in any of the products.

A third observation involved adding a 15 cc saline solution (0.9 gram per liter) insult to the liner side of new samples of the three different products in the crotch region and to the side. The point of insult was opposite location B as illustrated in FIG. 3. This location was selected so as to not to be directly over the backsheet graphics. This observation was repeated three times per brand using a new article each time. In each case, no visible change in the products was observed.

Based upon these observations, it appears that an insult of about 15 cc can be detected if located proximate to the indicator in the commercially available products. However, a 15 cc insult located slightly distant from the indicators did not provide a visual change. Finally, a 5 cc insult was not visually indicated in the commercial products even when located directly proximate the graphic.

Infants, e.g., greater than or equal to about 6 months old, produce insults of around 60 cc. However, newborns tend to have much smaller insults of around 10 to 15 cc, or even less. The absorbent articles disclosed herein (e.g., having an absorbent body comprising colorant) can be used to identify the existence of a very small volume of an insult (e.g., less than or equal to about 10 cc, or, more specifically, less than or equal to about 5 cc, and even down to an about 1 cc insult). The absorbent articles disclosed herein can detect “off target” insults as well. Finally, the absorbent articles disclosed herein can detect the presence of various substances that may be found in urine.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. An absorbent article, comprising:
a liquid permeable bodyside liner;
an outer cover; and
an absorbent body positioned between the bodyside liner and the outer cover;
wherein the absorbent body comprises a first colorant that becomes visible when wetted with urine so as to identify the presence of the urine.

2. The absorbent article of claim 1, wherein the first colorant becomes visible in the presence of about 1 cc to about 10 cc of urine.

3. The absorbent article of claim 1, wherein the first colorant becomes visible in the presence of about 1 cc to about 5 cc of urine.

4. The absorbent article of claim 1, wherein absorbent body has an area in the plane defined by the longitudinal direction and the lateral direction and the first colorant is disposed over greater than or equal to about 50% of the absorbent body area.

5. The absorbent article of claim 1, the absorbent body further comprising absorbent particles and absorbent fibers, wherein the colorant is a particulate and is intermixed with the superabsorbent particles and the absorbent fibers in the absorbent body.

6. The absorbent article of claim 1, wherein the first colorant becomes visible in the presence of a substance in the urine, and wherein the substance is selected from the group consisting of blood, protein, nitrates, ketones, glucose, leukocytes, urobilinogen, bilirubin and combinations comprising at least one of the foregoing substances.

7. The absorbent article of claim 6, wherein the first colorant is encapsulated in an encapsulant, and wherein the encapsulant is soluble in urine.

8. The absorbent article of claim 6, the absorbent body further comprising one or more second colorants, wherein the one or more second colorants becomes visible in the presence of a substance in the urine different than the substance in the urine that makes the first colorant visible.

9. An absorbent article, comprising:
a liquid permeable bodyside liner;
an outer cover;
an absorbent body located between the bodyside liner and the outer cover; and
a first colorant that becomes visible when wetted with urine containing a substance selected from the group consisting of blood, protein, nitrates, ketones, glucose, leukocytes, urobilinogen, bilirubin and combinations comprising at least one of the foregoing substances.

10. The absorbent article of claim 9, wherein the colorant is encapsulated in a material soluble in urine.

11. The absorbent article of claim 10, further comprising one or more second colorants, wherein the one or more second colorants becomes visible in the presence of a substance in the urine different than the substance in the urine that makes the first colorant visible.

12. An absorbent article, comprising: a colorant having a first state when the absorbent article is dry and a second state when the absorbent article is wetted with a volume of urine; and wherein the second state is visible and is indicative of the volume of the urine.

13. The absorbent article of claim 12, further comprising a liquid permeable bodyside liner, an outer cover and an absorbent body, wherein the absorbent body is located between the bodyside liner and the outer cover, and wherein the absorbent body comprises the colorant.

14. The absorbent article of claim 13, wherein the absorbent body further comprises cellulose fibers and superabsorbent particles, wherein the superabsorbent particles and colorant are dispersed between the cellulose fibers.

15. The absorbent article of claim 12, further comprising a surge material, and wherein an area of the second state is directly related to the volume of the liquid.

16. The absorbent article of claim 12, wherein the colorant comprises a dye encapsulated in a urine soluble material.

17. An absorbent article system, comprising:
a liquid permeable bodyside liner;
an outer cover;
an absorbent body comprising a colorant, the absorbent body located between the bodyside liner and the outer cover, wherein the colorant, when wetted, becomes visible and defines a visible area; and
a correlater useful in estimating a volume of liquid received by the absorbent body by comparing the visible area to the correlater.

18. The absorbent article system of claim 17, wherein the colorant is encapsulated in a urine soluble material.

19. The absorbent article system of claim 17, wherein the correlater is selected from the group consisting of a graph, a chart, a diagram, a table, a design, and combinations comprising at least one of the foregoing correlaters.
